(19) United States
(12) Patent Application Publication
(10) Pub. No.: US 2023/0206696 A1
(43) Pub. Date: Jun. 29, 2023

(54) MONITORING DEVICE

(71) Applicant: HUMA THERAPEUTICS LIMITED, London (GB)

(72) Inventors: Mert Aral, London (GB); Danoosh Vahdat, London (GB); Shahram Nikbakhtian, London (GB)

(21) Appl. No.: 17/921,531

(22) PCT Filed: Apr. 30, 2021
(86) PCT No.: PCT/GB2021/051054

§ 371 Date: Oct. 26, 2022

(30) Foreign Application Priority Data
Apr. 30, 2020 (GB) 2006410.1

(51) Int. Cl. G06V 40/20; G06T 7/246; G06V 10/10; G06V 20/40; G16H 40/67

(52) U.S. Cl. CPC G06V 40/25; G06T 7/251; G06V 10/17; G06V 20/41; G16H 40/67; G06T 2207/30196

(57) ABSTRACT

A monitoring system for monitoring the wellbeing of a user, the system comprising: one or more hand portable sensor devices each comprising at least one sensor for forming sensed data by sensing an activity characteristic of a user; and a processor configured to execute an algorithm in dependence on the sensed data to form an estimate of the wellbeing of the user; wherein at least one of the sensors is a camera and the processor is configured to analyse a video of the user captured by the camera to form the estimate of wellbeing.

This invention relates to a device for monitoring the wellbeing of a user.

Many electronic devices can now perform ongoing measurements of a user's physiology, For example, smart watches can monitor a user's heartrate, location and speed of movement. These metrics can provide information about the user's wellbeing. For example a reduction in a user's resting heart rate may indicate that the user's cardiovascular health is improving. On the other hand, a reduction in resting heart rate may be caused by medical conditions such as damage to heart tissue. The information derived from devices of this type is of limited value in a clinical context unless it is properly interpreted. Furthermore, many medical conditions cannot be directly sensed by devices of this type.

US 2014/0324443, US 2014/0074510, CN 109102888, US 2012/0108916, US 2015/0142332, US 2016/0275262, US 2012/0191469 and US 2018/0301220 disclose techniques for gathering information from a range of sensors/inputs and combining them to form an overall value intended to be indicative of a user's health or fitness.

It would be desirable to have a device that allows for better and/or more flexible assessment of a user's wellbeing.

According to one aspect of the present invention there is provided a monitoring system for monitoring the wellbeing of a user, the system comprising: one or more hand portable sensor devices each comprising at least one sensor for forming sensed data by sensing an activity characteristic of a user; and a processor configured to execute an algorithm in dependence on the sensed data to form an estimate of the wellbeing of the user; wherein at least one of the sensors is a camera and the processor is configured to analyse a video of the user captured by the camera to form the estimate of wellbeing.

One of the sensor devices may comprise the camera, that sensor device may further comprise a display and a processor, and that processor may be configured to: cause the display to present instructions for the user to execute a predetermined task; and cause the camera to capture a video whilst the user performs the task.

The task may be a physical activity.

The task may be one of: walking, running, bending, reaching or stretching.

The processor may be configured to analyse the video by: for each of multiple frames in the video, identifying a human in that frame and estimating the pose of the human; and estimating a change in pose between the frames.

The processor claimed in claim 1 may be configured to: for each of multiple frames in the video, identify a human in that frame, estimate the pose of the human, estimate a position of a first limb in that pose, estimate a position of a second limb in that pose; and estimate a maximum or minimum angle between those limbs over the multiple frames.

The processor may be configured to form the estimate of wellbeing by applying a set of predetermined weights to respective values derived from the sensors to form a set of weighted values, and aggregating the weighted values.

The estimate of wellbeing may be one of: an estimate of the physiological state of the user, an estimate of the mental state of the user, an estimate of the user's risk of injury and an estimate of the user's recovery from a medical procedure.

The processor may be configured to form the estimate of wellbeing by implementing a machine learned algorithm.

The one or more of the hand portable sensor devices may be a mobile phone.

The one or more of the hand portable sensor devices may be a wearable device provided with an attachment structure for attachment to a user.

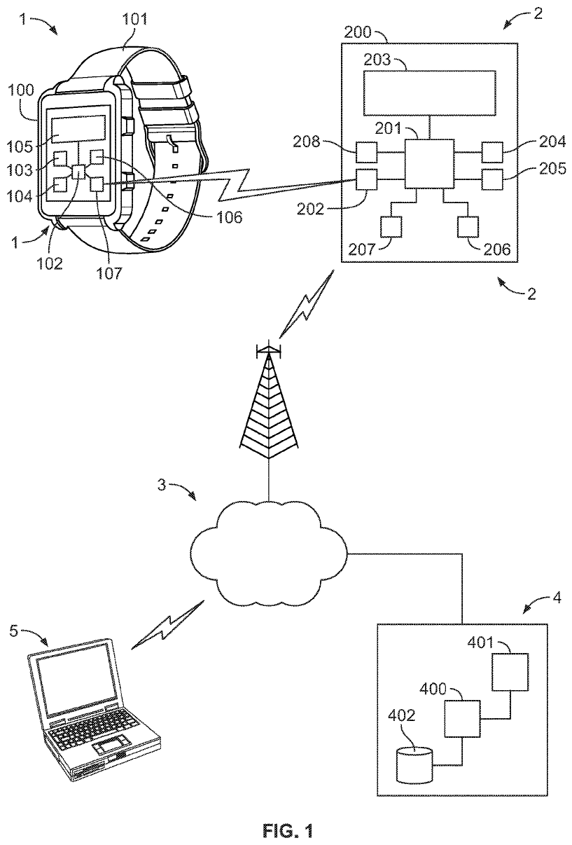

FIG. 1 shows a system for implementing the present invention. The invention may be implemented using all or just a subset of the elements shown in FIG. 1.

The system to be described below can develop an indicator of a user's wellbeing: for example their health, level of recovery from injury, propensity for injury, risk of death in a given time period or physical or mental ability. The indicator may conveniently be a number on a continuous or substantially continuous scale. The indicator may be formed using a predetermined algorithm. The algorithm may be formed in any convenient way, for example by manual training or supervised machine learning on previously acquired medical data.

Inputs to the algorithm for forming the estimate of wellbeing can be determined through answers to questions or measurements done by sensors. Sensors may sense short-term parameters (e.g. average heart rate over a period of 10 s, or present body mass) or long-term parameters (e.g. average number of hours of sleep over a week). A device may instruct a user to perform an action so that data can be sensed. These operations will be described in more detail below.

FIG. 1 shows a wearable device 1, in this case a smart watch; a local hub device 2, in this case a smartphone; a communications network 3; a server 4 and a terminal 5.

The wearable device 1 comprises a housing 100 which houses electronic components of the device. The housing is attached to a strap 101 which is sized for encircling the wrist of a user. In this example the wearable device is a wrist-wearable device but it could be worn in other locations on a user's body. For example it could comprise a clip for clipping to an item of clothing, or a chest strap. The housing has a processor 102, sensors 103, 104, a display 105, a memory 106 and a communications transceiver 107. The memory 106 stores in non-transient form program code which is executable by the processor 102 to cause it to perform the functions described of it herein. The processor can receive data from the sensors 103, 104, control the display 105 to display information as required and can transmit and receive information to and from other devices using the transceiver 107. The transceiver 107 may implement any suitable wired or wireless communication protocol. For example it could implement a wired USB protocol or a wireless protocol such as IEEE 802.11b, Bluetooth, ANT or a cellular radio protocol such as 3G, 4G or 5G. The sensors 102, 103 can sense characteristics that are relevant to the wellbeing of a user/wearer of the device 1. Examples of such characteristics are given below.

The hub device 2 is suitable for being carried by a user. It serves to collect data from the wearable device 1 and process that data and/or transmit it to server 4. It comprises a housing 200 which houses electronic components of the device. The device comprises a processor 201, a first transceiver 202, a display 203, a memory 204, sensors 205, 206 and a second transceiver 207. The display may be a touchscreen display that is capable of receiving user input as well as displaying information. Alternatively, the device may additionally comprise a user input device 208 such as a keypad. The memory 204 stores in non-transient form program code which is executable by the processor 201 to cause it to perform the functions described of it herein. The processor can receive data from the sensors 205, 206, control the display 203 to display information as required, receive user input from the display and/or keypad 208 and can transmit and receive information to and from other devices using transceivers 202, 207. Each transceiver 107 may independently implement any suitable wired or wireless communication protocol. For example each could implement a wired USB protocol or a wireless protocol such as IEEE 802.11b, Bluetooth, ANT or a cellular radio protocol such as 3G, 4G or 5G. In a convenient embodiment, both transceivers implement wireless protocols and transceiver 202 implements a shorter range protocol than transceiver 207. For example, transceiver 202 may implement an ISM band protocol such as IEEE 802.11b or Bluetooth and transceiver 207 may implement a cellular radio protocol. Transceiver 207 is communicatively coupled to network 3.

Device 2 is preferably hand portable. That is, it can be carried readily by a user. It may have a greatest dimension less than 20 cm. It may weigh less than 1 kg or 500 g or 200 g.

Network 3 serves to interconnect devices 2, 4 and 5. It may comprise wireless and/or wired communication links. It may comprise a publicly accessible network such as the internet. It may comprise a cellular radio network.

The server 4 is communicatively coupled to network 3. The server may analyse data collected by devices 1 and 2. It may also aggregate data from multiple such devices used by different users and process the aggregated data. It comprises a processor 400, a memory 401 and a database 402. The memory 401 stores in non-transient form program code which is executable by the processor 400 to cause it to perform the functions described of it herein. The database 402 stores historic data collected by devices 1 and 2 and optionally other like devices.

Terminal 5 is communicatively coupled to network 3. Terminal 5 may be used to communicate with device 2 and/or with server 4 to retrieve information and/or to configure the system, e.g. to set an algorithm for analysing data from a user or to set performance targets for a user.

A practical system may omit some of the elements in FIG. 1, and may have additional elements. Some illustrative examples will be given. Device 1 may communicate directly with network 3, in which case device 2 may be omitted. Device 1 may perform local data analysis and present the results to a user, in which case the other devices and the network are not needed. Device 2 may perform local data analysis and present the results to a user, in which case devices 4 and 5 and the network 3 are not needed. Device 5 may communicate over network 3 directly to device 1 or 2, to configure such a device or to receive data collected by it.

The sensors 103, 104, 205, 206 can sense characteristics or parameters that are relevant to the wellbeing of a user/wearer of the device 1. One or more of the sensors of device 1 may sense the same parameter as a sensor of device 2. Alternatively, all the sensors may sense different parameters. The parameters may be environmental parameters that are independent of the physiology of the wearer of the device 1 or a user of device 2, or parameters that indicate the position or motion of such a wearer or user, or parameters that indicate a physiological characteristic of such a wearer or user. Some non-limiting examples will now be given. Examples of environmental parameters include external air pressure, external temperature, external humidity noise or the level of ambient light. These may be captured by suitable sensors, e.g. a pressure sensor, a temperature sensor and so on. Examples of positional/motion parameters include position (which may be derived e.g. from a satellite location system or from a short-range radio locationing system), altitude, speed, direction, the orientation of the device in question (1 or 2) and a pattern of motion of the device (indicating for example a particular gait or type of motion of a person carrying or wearing the device, which may be dependent on where the device in question is worn or carried). These may be captured by suitable sensors, e.g. one or more accelerometers, a gravity sensor, a satellite locationing device and so on. Examples of physiological parameters include heart rate, breathing rate, blood pressure, blood oxygen concentration and body temperature. These may be captured using suitable physiological sensors. In addition, one of the sensors on either device 1 or 2 may be a camera capable of capturing still images or video of scenes external to the device, or a button or other contact user input device for capturing keypresses or gestures of a user.

Each sensor may sense data continuously or discontinuously. They may sense data at predetermined intervals or times of the day, or when commanded by a user or terminal 5, or in response to a predetermined condition sensed by another of the sensors. Each sensor may sense data without any specific activity being required on the part of a user. Alternatively, one or more sensors may operate to sense data when signalled by a user to do so and/or when a user is performing a predetermined action. In one example, data may be sensed when a user is performing an action that has been predetermined for providing information as to a physiological state. Processor 102/201 may cause the respective display 105/203 to display a instruction to a user to perform an action. The instruction may include information explaining how to perform the action. The user can perform the action in response to that prompt, and one or more sensors of the respective device can capture data as the action is performed. For instance, the action may be to press a button in response to a prompt on the screen, and the timings of the appearance of the prompt and of the pressing of the button may be recorded so as to gauge the user's reaction time. Or the instruction may be to throw and catch a ball and the device in question may record a video of the user performing that action.

Device 1 or 2 may be configured to present a question to a user by means of display 105, 203 or by audio means (not shown). The question may, for example ask about the user's subjective wellbeing or whether they have adhered to a wellness programme (e.g. taking medication). The user may answer the question by providing input to the device, e.g. by pressing buttons, using a touch screen or responding by voice. That input may constitute sensed input.

The sensors 103, 104 may sense data autonomously and transmit that data to processor 102, or they may sense data under the command of processor 102. When processor 102 has received data it may cache it in memory 106. Processor 102 may process the sensed data, e.g. to filter, compress or analyse it. Processor 102 may then cause transceiver 107 to transmit the data to transceiver 202, or directly via network 3 to server 4 and/or terminal 5. That transmission may be done immediately the data is collected, or it may be done later.

The sensors 205, 206 may sense data autonomously and transmit that data to processor 201, or they may sense data under the command of processor 201. When processor 201 has received data it may cache it in memory 204. Processor 201 may also receive data via transceiver 202 that has been sensed by device 1. Processor 201 may process the sensed data, e.g. to filter, compress or analyse it. Processor 102 may then cause transceiver 207 to transmit the data to via network 3 to server 4 and/or terminal 5.

It may be assumed that the user of a device 1, 2 is constant unless the system is informed of a change. Alternatively, a user may be identified by logging into a device and providing security credentials.

When sensed data is received at server 4, the server stores the data in datastore 402. It can also process the data, e.g. to filter, compress or analyse it. Some examples of the forms of analysis that may be performed include (i) comparing the data with data sensed at an earlier time for the same user: this can indicate trends in the behaviour or wellbeing of that user; (ii) comparing the data with data sensed for other users: this can indicate the user's state relative to an average; (iii) comparing the data with a predetermined algorithmic model, which may be a machine learning model: this can indicate the user's state relative to a theoretical state. The results of such analysis can be transmitted to device 1, 2 or 4 for viewing by a user.

One convenient way in which the collected data can be used is as follows: 1. At a first time, data is collected by one or more of sensors 103, 104, 205, 206. 2. That data is forwarded to server 4. The processor of server 4 analyses the sensed data by comparing it to data previously received in respect of the same user, and by executing a predetermined algorithm on the data. The output of the algorithm will be termed analysis data. The significance of the analysis data and the manner in which it is formed will be discussed below. 3. The analysis data is passed to device 5, for review by a healthcare professional, and/or to device 1 or 2 for review by the end-user. The healthcare professional may advise the end-user in dependence on the analysis data. The end-user may adapt their behaviour in dependence on the analysis data.

The algorithm that forms the analysis data could be implemented using a processor of device 1 or device 2. It could be distributed between processors at multiple locations. Server 4 may be remote from devices 1 and 2. Devices 1 and 2 may be within 1 m or 2 m of each other when sensing is performed. The maximum suitable distance will depend on the communication mechanism (if any) employed between the devices and whether either device can buffer sensed data before transmitting it.

The analysis data may be formed by synthesising information derived from any of the following: (i) the sensed data, (ii) previous sensed data in respect of the same user, (iii) previous sensed data in respect of other users, (iv) a model of physiological performance, (v) other data held by server 4 or another data store relating to the user (e.g. the user's age or medical history).

Some non-limiting examples of data that may be input into an algorithm for estimating a score as described herein are: sociodemographics, educational attainment, behaviour, nutritional intake, lifestyle factors, medication use, clinical history, gender, age, educational qualifications, ethnicity, previous diagnoses of cancer, coronary heart disease, type 2 diabetes or COPD, smoking history, blood pressure, Townsend deprivation index, BMI, FEV1, waist circumference, blood pressure parameters, skin tone, smoking status, age, prior cancer diagnosis, prescription of digoxin, residential air pollution, average sleep duration, resting heart rate, alcohol consumption, self-rated health, reaction time and waist-to-height ratio. Any one or more of the above parameters, together optionally with other parameters may be used.

In one example, the algorithm may store a set of weights. Multiple parameters from the list above may be multiplied by respective weights and then those products may be added together to form an aggregated score acting as the analysis data. The weights may be manually generated or derived from a machine learning process. In another example, the algorithm may apply pre-stored logic instead or in addition to weighting. The logic may be manually generated and/or derived from a machine learning process. In these ways, the algorithm can, in effect, combine data including that sensed by devices 1, 2, to form a single overall value or score. Having a single overall value can make it easier for a healthcare professional or an end-user to readily appreciate the end-user's level of wellbeing.

The algorithm used to form the score may be such that the score is on a substantially continuous scale: for example a whole number that can take value in the range from 0 to 100.

One possible set of inputs that may be used comprises five or more of, and more preferably six or more of: resting heart rate, average hours of sleep, waist-to-height ratio, self-rated health, smoking level, alcohol consumption and reaction time. For each such parameter a value may be allocated based on the subject's status in a predetermined range. Then those values may be weighted by multiplying by a weight, and the weights may be added together to yield the wellbeing score. Examples of the relative weights that may be used are any two or more, three or more, four or more, five or more, six or more or seven of the following:

Resting heart rate: a value in the range 5 to 10

Sleep: a value in the range 8 to 13

Waist-to-height ratio: a value in the range 8 to 13

Self-rated health: a value in the range 29 to 35

Smoking level: a value in the range 10 to 14

Alcohol consumption: a value in the range 15 to 23

Reaction time: a value in the range 4 to 8

Reaction time may be measured by a portable device using the protocols described herein.

If a score or aggregated metric is to be formed by a machine learned algorithm, the algorithm can be trained using training data sensed in respect of a population of users, and ground truth data that represents a desired outcome for the score in respect of each of those users. Then a suitable machine learning process can be performed to train a machine learning algorithm to generate the ground truth data or an approximation of it in response to the training data.

Examples of machine learning algorithms that can be used to develop an algorithm for forming the metric are supervised machine learning classifiers such as K-nearest neighbour (KNN) classifier and the supervised vector machine (SVM) classifier. These may be used to select the preferred hyper-parameters using (e.g.) 10-fold cross validation on suitable training and validation sets of historic data on individuals' medical histories.

The score may indicate any of a number of estimates of wellbeing, or a combination thereof. Some examples include:

an overall estimate of a user's wellbeing;

an estimate of the user's wellbeing in a specific respect: for example their level of flexibility at a particular joint or their propensity to depression;

the user's level of adherence to a prescribed regime: for example taking medication at predetermined times, or taking exercise, or not smoking;

the user's level of recovery from a medical procedure: for example the user's level of recovery from arthroplasty.

As indicated above, one of the aspects of sensed data that can be processed by the algorithm is video or still image data captured by a camera of device 1 or 2 showing the user performing an action. The device in question may display a prompt to a user to perform an action. That prompt may be displayed at a fixed time of day, or at a random time. The user can then photograph or video themselves performing the action. The still image or the video can then be analysed locally on the respective device, on the other of the devices 1, 2, at server 4, or manually by a user of terminal 4. When the video or image is analysed automatically, image recognition software may be used to process the image or picture and extract relevant information from it. Image recognition software of that type is well known. In one example, it may be machine learning software that is trained to identify the required information. If the automatic analysis software cannot identify the action in the video or still image with greater than a predetermined level of probability, the user may be prompted to perform the action again. That might happen if the user has failed to perform the action, performed a different action, or pointed the device's camera in the wrong direction. The action may be a physical activity. Some examples of the actions that the user may be prompted to perform, and the data that may be identified from an image or video of that action are:

throwing and catching a ball—this may indicate the user's level of balance and coordination;

reading text aloud—this may indicate aspect of the user's cognitive state;

bending at a joint—this may indicate the user's level of flexibility at that joint;

walking—this may indicate the user's level of balance and mobility. Other examples include running, reaching and stretching. In one method of analysis, an image recognition algorithm is used to process the image or video to for each of multiple frames in the video, identifying a human in that frame and estimating the pose of the human. The algorithm may estimate the joint positions of a human shown in the video. See, e.g. “Joint Action Recognition and Pose Estimation From Video”, Nie et al., Conference on Computer Vision and Pattern Recognition 2015. From those joint positions the human's pose can be estimated, in accordance with a stick figure model. Then the pose or motion (i.e. the change of pose over time) can be estimated. Another factor that may be estimated is the maximum or minimum angle achieves at a predetermined joint. The pose can be compared to one or more models, and from any deviation from the model estimates can be made of factors such as the user's ability to balance, flexibility or posture. In another method of analysis, a sound analysis and/or voice recognition algorithm can be used to process sound recorded in a video by a microphone sensor, or in a simple audio file. That analysis may provide information about the clarity of a user's speech, variations in tone of voice and so on.

In some examples given above, the outcome of the analysis algorithm is an indication of a sensed factor relating to the user. In a further enhancement, one or more such factors and/or original sensed data may be used to estimate a status of the user. For example, from information about the user's age (which could be entered into the system), the frequency with which the user climbs stairs (which could be derived from gait monitoring using one or more accelerometers) and the user's level of balance (which could be estimated from video analysis as described above) an estimate of the user's risk of falling could be estimated. In another example, from information about the user's level of flexibility at the knee (derived from video analysis), the user's level of activity (derived from accelerometers) and the user's level of adherence to a stretching or strengthening regime (derived from the user's answers to questions posed by device 1 or 2) the user's need for physiotherapy intervention following arthroplasty could be estimated. In each case, a score may be formed by weighting sensed data and/or data formed in dependence on sensed data From information of that nature a healthcare professional can assess the need for intervention to assist the user.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A monitoring system for monitoring the wellbeing of a user, the system comprising:
one or more hand portable sensor devices each comprising at least one sensor for forming sensed data by sensing an activity characteristic of the user; and
a processor configured to execute an algorithm in dependence on the sensed data to form an estimate of the wellbeing of the user,
wherein at least one of the sensors is a camera and the processor is configured to analyze a video of the user captured by the camera to form the estimate of the wellbeing of the user.

2. A monitoring system as claimed in claim 1, wherein one of the sensor devices comprises the camera, a display, and a second processor, the second processor being configured to:
cause the display to present instructions for the user to execute a predetermined task; and
cause the camera to capture the video whilst the user performs the task.

3. A monitoring system as claimed in claim 2, wherein the task is a physical activity.

4. A monitoring system as claimed in claim 3, wherein the task is at least one of walking, running, bending, reaching, or stretching.

5. A monitoring system as claimed in claim 1, wherein the processor is configured to analyze the video by:
for each of multiple frames in the video, identifying the user in that frame and estimating the pose of the user; and
estimating a change in pose between the frames.

6. A monitoring system as claimed in claim 1, wherein the processor is configured to:
for each of multiple frames in the video, identify the user a human in that frame, estimate a pose of the user, estimate a position of a first limb in the pose, estimate a position of a second limb in the pose; and
estimate a maximum or minimum angle between the first limb and the second limb over the multiple frames.

7. A monitoring system as claimed in claim 1, wherein the processor is configured to form the estimate of the wellbeing of the user by applying a set of predetermined weights to respective values derived from the sensors to form a set of weighted values, and aggregating the weighted values.

8. A monitoring system as claimed in claim 1, wherein the estimate of the wellbeing of the user is at least one of an estimate of a physiological state of the user, an estimate of a mental state of the user, an estimate of the user's risk of injury, or an estimate of the user's level of recovery from a medical procedure.

9. A monitoring system as claimed in claim 1, wherein the processor is configured to form the estimate of the wellbeing of the user by implementing a machine learning algorithm.

10. A monitoring system as claimed in claim 1, wherein at least one of the one or more hand portable sensor devices is a mobile phone.

11. A monitoring system as claimed in claim 1, wherein at least one of the one or more hand portable sensor devices is a wearable device provided with an attachment structure for attachment to the user.
